(19) United States
(12) Patent Application Publication
(10) Pub. No.: US 2025/0017674 A1
(43) Pub. Date: Jan. 16, 2025

(54) SURGICAL ROBOTIC SYSTEM AND METHOD FOR OPTICAL MEASUREMENT OF END EFFECTOR PITCH, YAW, AND JAW ANGLE

(71) Applicant: Covidien LP, Mansfield, MA (US)

(72) Inventors: Daniel A. Joseph, Golden, CO (US); Christopher T. Brown, Boulder, CO (US); Donavon W. Facey, Lafayette, CO (US); Kiana Martin, Portage, MI (US); Ashley B. Withnell, Niwot, CO (US)

(21) Appl. No.: 18/766,857

(22) Filed: Jul. 9, 2024

Related U.S. Application Data
(60) Provisional application No. 63/526,935, filed on Jul. 14, 2023.

(51) Int. Cl. A61B 34/37; A61B 34/30; A61B 90/00

(52) U.S. Cl. CPC A61B 34/37; A61B 90/361; A61B 2034/301; A61B 2034/305

(57) ABSTRACT

A surgical robotic system includes an instrument having a shaft defining a longitudinal axis and an end effector pivotable relative to the shaft at a yaw angle, the end effector including a pair of jaws pivotable at a pitch angle and openable to a jaw angle. The system also includes a first imaging device configured to obtain a first image of the end effector along a first axis; a second imaging device configured to obtain a second image of the end effector along a second axis; and a third imaging device configured to obtain a third image of the end effector along a third axis, wherein each of the first, second, and third axes are transverse relative to each other.

### CROSS-REFERENCE TO RELATED APPLICATIONS

This application claims the benefit of, and priority to, U.S. Provisional Patent Application Ser. No. 63/526,935 filed on Jul. 14, 2023. The entire contents of the foregoing application is incorporated by reference herein.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

### SUMMARY

According to one embodiment of the present disclosure, a surgical robotic system is disclosed. The system includes an instrument having a shaft defining a longitudinal axis and an end effector pivotable relative to the shaft at a yaw angle, the end effector including a pair of jaws pivotable at a pitch angle and openable to a jaw angle. The system also includes a first imaging device configured to obtain a first image of the end effector along a first axis; a second imaging device configured to obtain a second image of the end effector along a second axis; and a third imaging device configured to obtain a third image of the end effector along a third axis, wherein each of the first, second, and third axes are transverse relative to each other.

Implementations of the above embodiment may include one or more of the following features. According to one aspect of the above embodiment, the first imaging device may be configured to obtain the first image of the end effector for determining the yaw angle, the second imaging device may be configured to obtain the second image of the end effector for determining one of the pitch angle or the jaw angle, and the third imaging device may be configured to obtain the third image of the end effector for determining one of the pitch angle or the jaw angle. The end effector may be pivotable relative to the shaft about a first pin defining a first pivot axis. The end effector may include a pair of jaws pivotable about a second pin defining a second pivot axis. The system may further include an image processing device configured to process the first image, the second image, and the third image and to calculate the yaw angle, the pitch angle, and the jaw angle. The first imaging device may be disposed coaxially with the first pivot axis that is perpendicular to the longitudinal axis. The second imaging device may be disposed coaxially with the longitudinal axis. The third imaging device may be disposed perpendicular to the longitudinal axis and the first pivot axis. Each of the first imaging device, the second imaging device, and the third imaging device may be an optical comparator. The system may also include an imaging assembly configured to couple to the surgical instrument and to secure the first imaging device, the second imaging device, and the third imaging device relative to the end effector. The imaging assembly may further include a first mount defining a plane that is aligned with the longitudinal axis and a second mount coupled to the first mount, the second mount defining a second plane that is transverse to the first plane.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an aspect of the present disclosure;

FIG. 6 is a perspective view, with parts separated, of an instrument drive unit and a surgical instrument according to an embodiment of the present disclosure;

FIG. 7 is a top, perspective view of an end effector, according to an embodiment of the present disclosure, for use in the surgical robotic system of FIG. 1;

FIG. 8 shows the end effector in various configurations according to an embodiment of the present disclosure;

FIG. 9 shows an imaging assembly for optical measurement of end effector pitch, yaw, and jaw angle according to an embodiment of the present disclosure;

FIG. 10 shows an image of the end effector captured by an imaging device of the imaging assembly of FIG. 9; and

FIG. 11 shows an imaging assembly for optical measurement of end effector pitch, yaw, and jaw angle according to another embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices. The input is processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands and to generate a torque commands for activating one or more actuators of the robotic arm, which would, in turn, move the robotic arm in response to the movement commands.

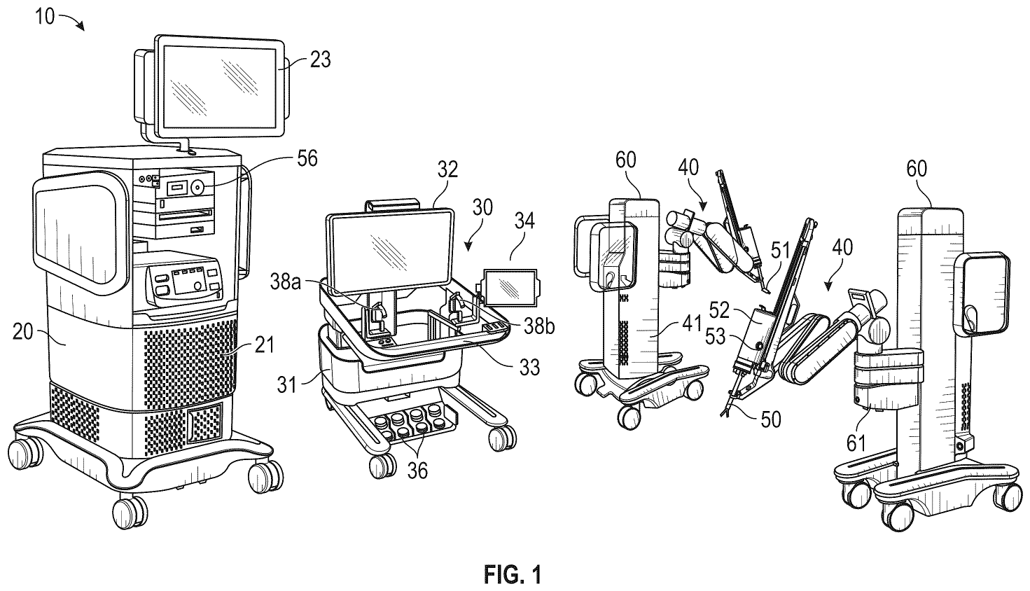

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a image processing device 56, which may be disposed within the control tower 20. The image processing device 56 may be any computing device as described below configured to receive the video feed from the endoscopic camera 51 and output the processed video stream.

The surgeon console 30 includes a first display 32, which displays a video feed of the surgical site provided by camera 51 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

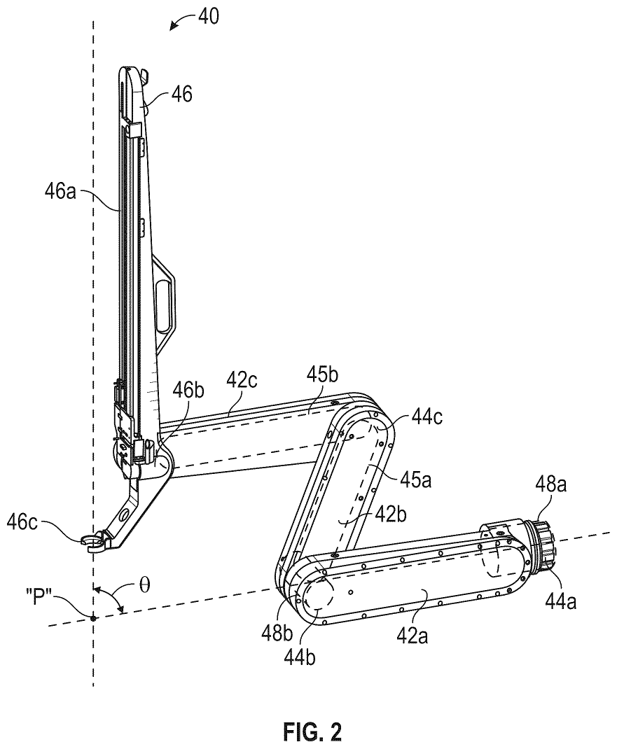

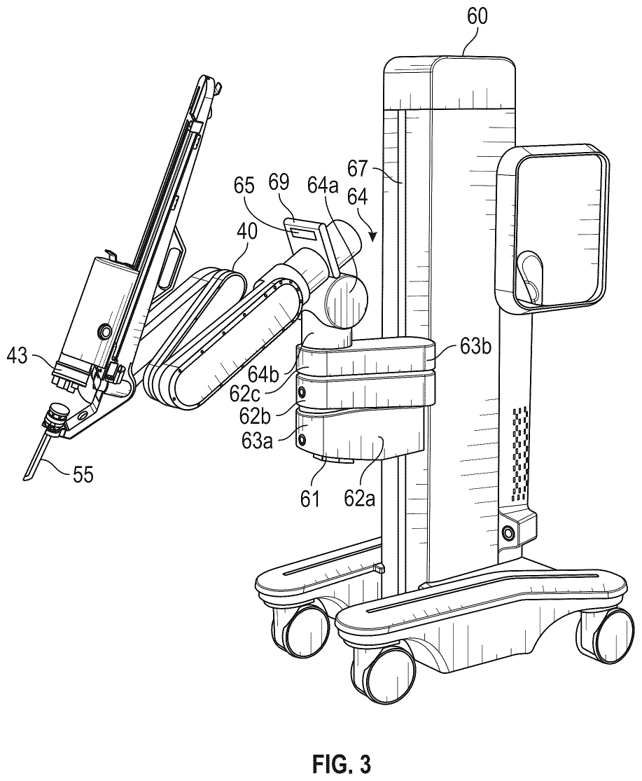

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 200 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

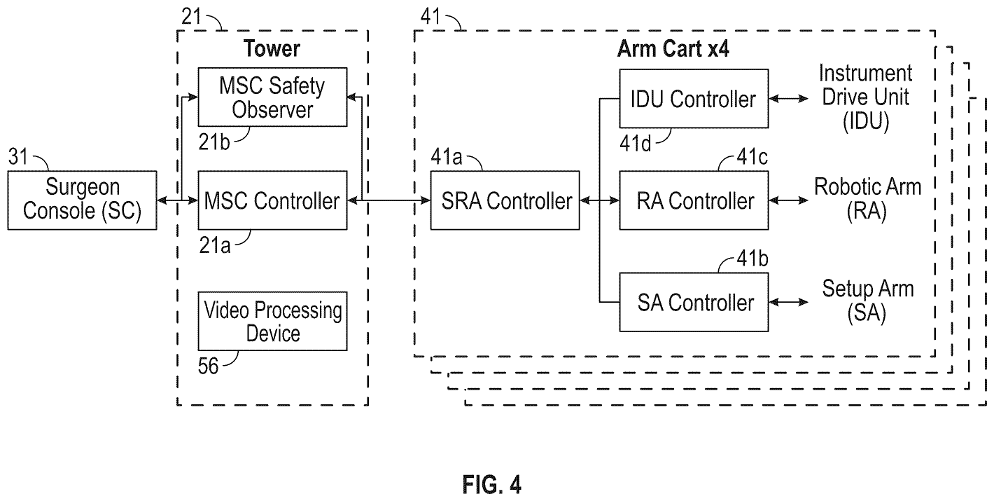

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

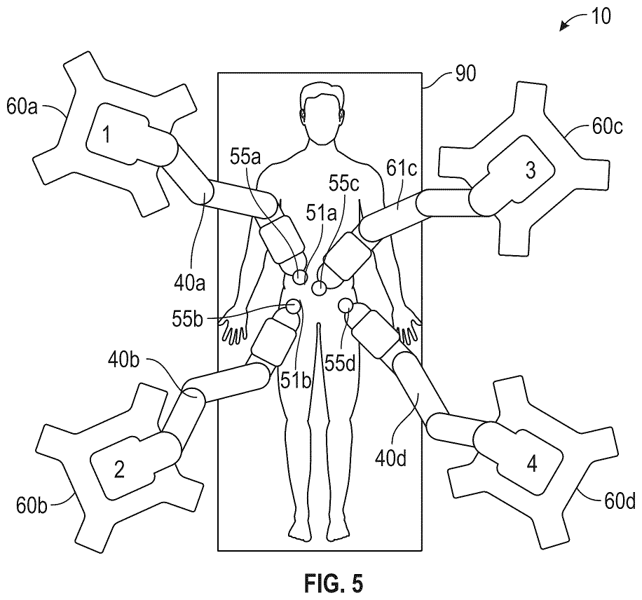

With reference to FIG. 5, the surgical robotic system 10 is setup around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

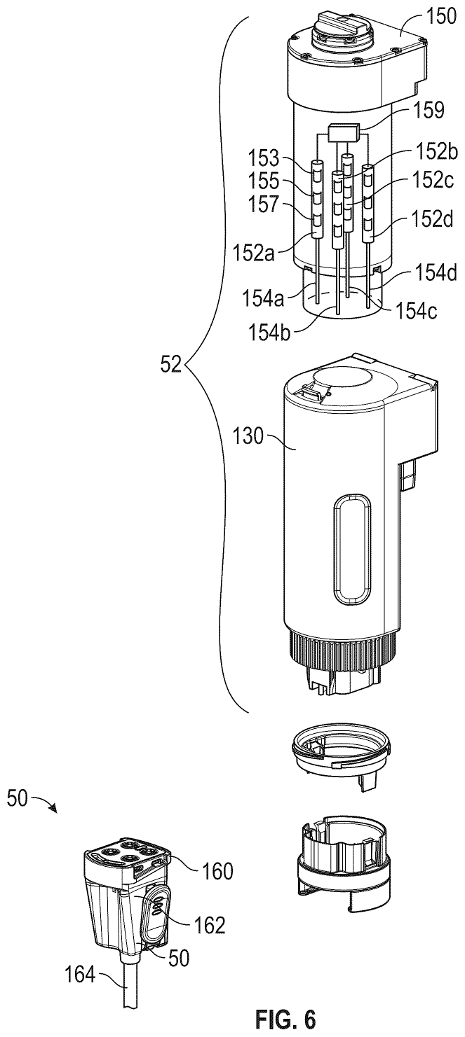

With reference to FIG. 6, the IDU 52 is shown in more detail and is configured to transfer power and actuation forces from its motors 152a, 152b, 152c, 152d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

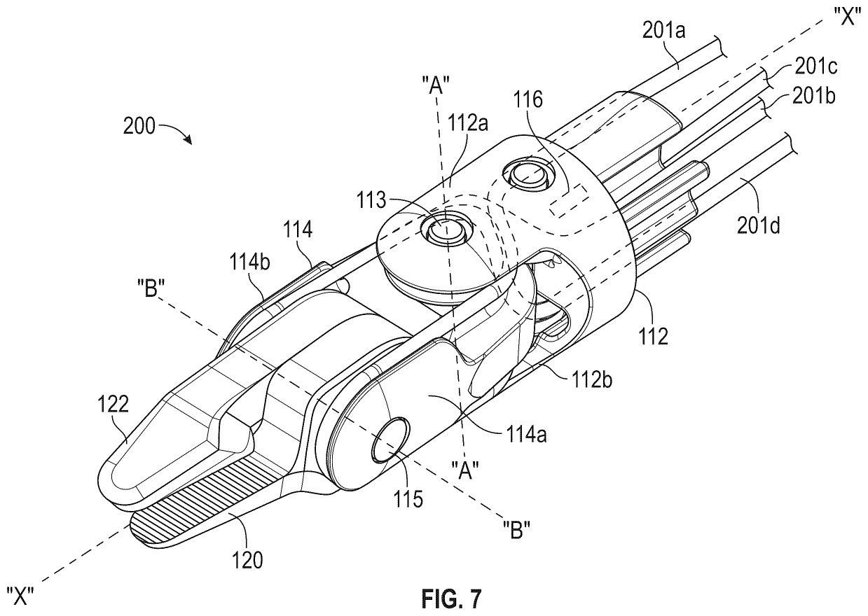

The IDU 52 includes a motor pack 150 and a sterile barrier housing 130. Motor pack 150 includes motors 152a, 152b, 152c, 152d for controlling various operations of the instrument 50. The instrument 50 is removably couplable to IDU 52. As the motors 152a, 152b, 152c, 152d of the motor pack 150 are actuated, rotation of the drive transfer shafts 154a, 154b, 154c, 154d of the motors 152a, 152b, 152c, 152d, respectively, is transferred to the drive assemblies of the instrument 50. The instrument 50 is configured to transfer rotational forces/movement supplied by the IDU 52 (e.g., via the motors 152a, 152b, 152c, 152d of the motor pack 150) into longitudinal movement or translation of the cables or drive shafts to effect various functions of an end effector 200 (FIG. 7).

Each of the motors 152a, 152b, 152c, 152d includes a current sensor 153, a torque sensor 155, and a position sensor 157. For conciseness only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by motor 152a. Position sensor 157 may be any device that provides a sensor signal indicative of the number of rotations of the motor 152a, such as a mechanical encoder or an optical encoder. Parameters which are measured and/or determined by position sensor 157 may include speed, distance, revolutions per minute, position, and the like. The sensor signals from sensors 153, 155, 157 are transmitted to the IDU controller 41d, which then controls the motors 152a, 152b, 152c, 152d based on the sensor signals. In particular, the motors 152a, 152b, 152c, 152d are controlled by an actuator controller 159, which controls torque outputted and angular velocity of the motors 152a, 152b, 152c, 152d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

With reference to FIG. 6, instrument 50 includes an adapter 160 having a housing 162 at a proximal end portion thereof and an elongated shaft 164 that extends distally from housing 162. Housing 162 of instrument 50 is configured to selectively couple to IDU 52 of robotic, to enable motors 152a, 152b, 152c, 152d of IDU 52 to operate the end effector 200 of the instrument 50. Housing 162 of instrument 50 supports a drive assembly that mechanically and/or electrically cooperates with motors 152a, 152b, 152c, 152d of IDU 52. Drive assembly of instrument 50 may include any suitable electrical and/or mechanical component to effectuate driving force/movement.

The surgical instrument also includes an end effector 200 coupled to the elongated shaft 164. The end effector 200 may include any number of degrees of freedom allowing the end effector 200 to articulate, pivot, etc., relative to the elongated shaft 164. The end effector 200 may be any suitable surgical end effector configured to treat tissue, such as a dissector, grasper, sealer, stapler, etc.

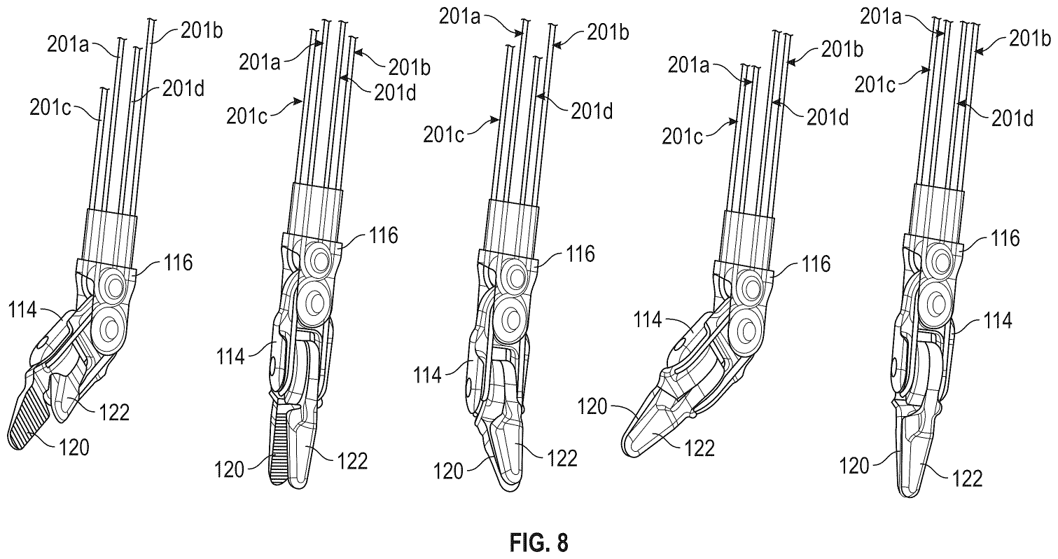

As shown in FIGS. 7 and 8, the end effector 200 may include a pair of opposing jaws 120 and 122 that are movable relative to each other. In embodiments, the end effector 200 may include a proximal portion 112 having a first pin 113 and a distal portion 114. The end effector 200 may be actuated using a plurality of cables 201a-d routed through proximal and distal portions 112 and 114 around their respective pulleys 112a, 112b, 114a, 114b, which are integrally formed as arms of the proximal and distal portions 112 and 114. Each of the cables 201a-d is actuated by a respective motor 152a-d via corresponding couplers disposed in adapter 160. In embodiments, the end effector 200, namely, the distal portion 114 and the jaws 120 and 122, may be articulated about the axis “A-A” to control a yaw angle of the end effector with respect to a longitudinal axis “X-X”. The distal portion 114 includes a second pin 115 with a pair of jaws including a first jaw 120 and a second jaw 122 pivotably coupled to the second pin 115. The jaws 120 and 122 are configured to pivot about an axis “B-B” defined by the second pin 115 allowing for controlling a pitch angle of the jaws 120 and 122 as well as opening and closing the jaws 120 and 122. The yaw, pitch, and jaw angles between the jaws 120 and 122 as they are moved between open and closed positions are controlled by adjusting the tension and/or length and direction (e.g., proximal or distal) of the cables 201a-d as shown in FIG. 8. The end effector 200 also includes a cable displacement sensor 116 configured to measure position of the cables 201. Thus, the end effector 200 may have three degrees of freedom, yaw, pitch, and jaw angle between jaws 120 and 122.

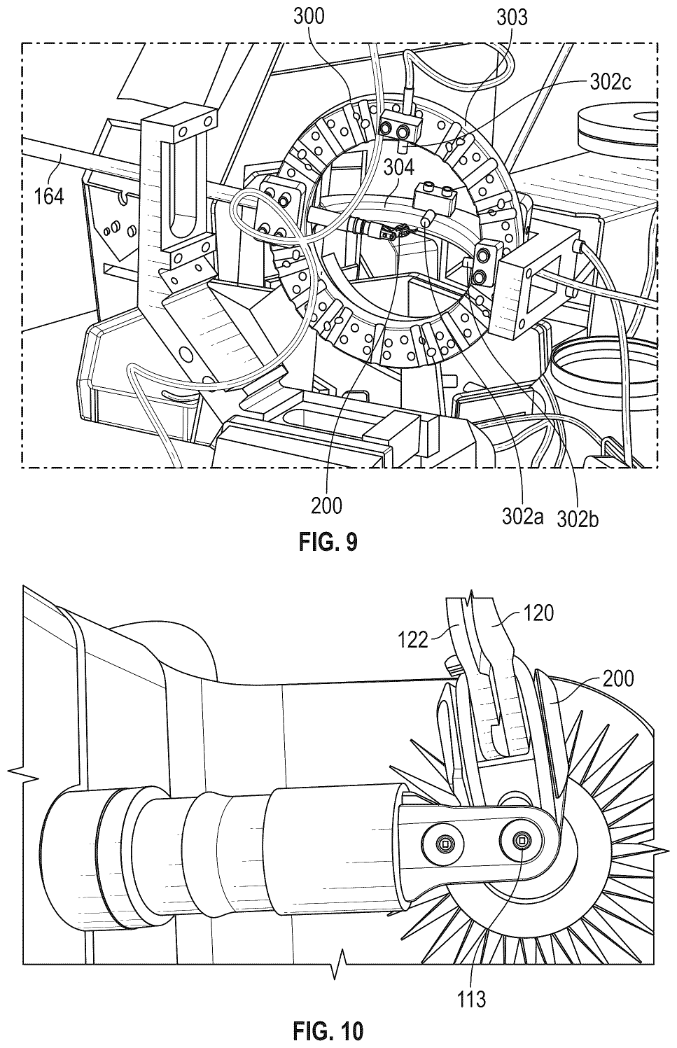

FIG. 9 shows an imaging assembly 300 for optical measurement of yaw, pitch, and jaw angle of the end effector 200. While the end effector 200 is shown as a grasper in FIGS. 7 and 8, the imaging assembly 300 may be used to image other suitable end effectors such as shears as shown in FIGS. 9-11. The instrument 50, and in particular, the end effector 200 is configured to be inserted into the imaging assembly 300 to allow for measurement of the instrument 50, and in particular, determining yaw, pitch, and jaw angle of the end effector 200 based on captured images.

The imaging assembly 300 includes a plurality of imaging devices 302a-c, which may be a video camera or an optical comparator, which may be a telecentric measurement device, such as TM-X5000 Series available from Keyence Corporation of Osaka, JP. Such devices utilize an image sensor disposed opposite a light source, e.g., laser, backlighting the object being imaged. This arrangement allows for imaging the profile of the end effector 200 and the jaws 120 and 122 providing for accurate optical measurement of displacement. In addition, optical measurement provides more accurate and faster calculations of position of mechanical components than encoder sensors due to direct measurement of the jaw position. Furthermore, these measurements use less processing power as there is no need to extrapolate jaw position based on the position of the motor.

The imaging devices 302a-c provide images of the end effector 200 to the image processing device 56, which determines yaw, pitch, and jaw angle. With reference to FIG. 10, the image processing device 56 is configured to identify a plurality of points of the imaged end effector 200 corresponding to the pivot pins 113, 115, and a heel of the shears (i.e., jaws 120 and 122). The image processing device 56 then generates connecting lines, which are then used to determine the angles of the end effector 200 and the jaws 120 and 122. The determined angles may be used to calibrate the end effector 200 by correlating determined angles with kinematic data of the IDU 52 controlling the instrument 50/end effector 200.

The imaging assembly 300 includes a first mount 303 defining a first plane that is parallel to a longitudinal axis defined by the elongated shaft 164 of the instrument 50. The imaging assembly 300 also includes a second mount 304 coupled to the first mount 303. The second mount 304 defines a second plane that is transverse to the first plane of the first mount 303 and the longitudinal axis of the instrument 50. The first imaging device 302a is mounted on the second mount 304 and its imaging axis is coaxial with the pivot axis defined by the first pin 115. The first imaging device 302a is configured to measure yaw angle, θ, i.e., between proximal and distal portions 112 and 114.

The second and third imaging devices 302b and 302c are coupled to the first mount 300. The second imaging device 302b is mounted in line with the longitudinal axis and is configured to measure jaw angle relative to centerline of the end effector 200 when the yaw angle of the end effector is outside −45° and/or +45° relative to the longitudinal axis.

The third imaging device 302c measures jaw angles when the yaw angle of the end effector is between −45° and/or +45° relative to the longitudinal axis. This third imaging device 302c is mounted perpendicular to the longitudinal axis and perpendicular to the proximal pivot axis of the first pivot pin 115 to view the jaws 120 and 122 as they move.

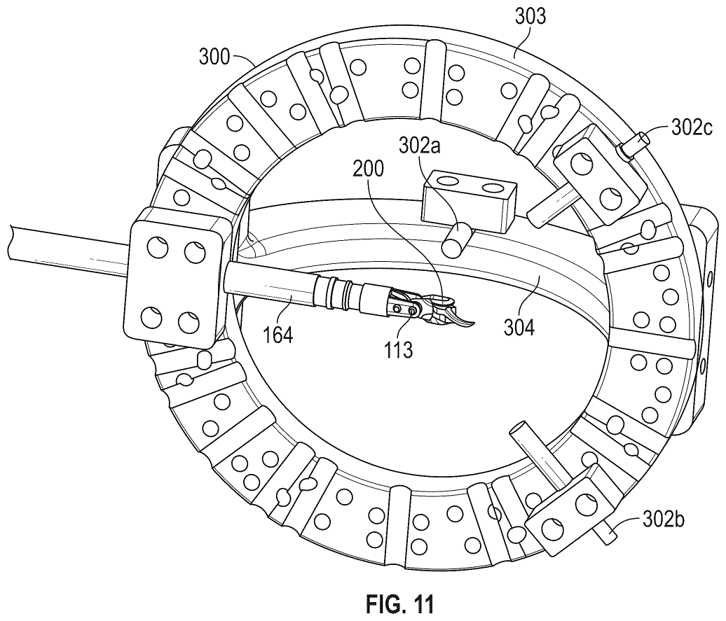

With reference to FIG. 11, in embodiments, the second and third imaging devices 302b and 302c may be mounted at about 45° off the longitudinal axis of the instrument 50. This allows for insertion of the end effector 50 into the access port 55 to enable mechanical limit calibration. The first and second mounts 303 and 304 may also include a plurality of depressions for securing the imaging devices 302a-c at any suitable angle for enabling imaging of the end effector 200.

Position of the jaws 120 and 122 estimated based on the rotational position of the motors 152a-d that move the jaws 120 and 122 may not be accurate due to multiple mechanical components interposed between the motors 152a-d and the jaws 120 and 122 (i.e., only the movement of the motors 152a-d is measured, rather than the jaws 120 and 122 themselves). In contrast, the disclosed optical measurement system for determining yaw, pitch, and jaw angle of jaws 120 and 122 provides an accurate and direct measurement of the position of the jaws 120 and 122 and corresponding angles.

These measurements may be used for a variety of purposes in controlling the movement of the jaws 120 and 122, such as more accurate testing of the instrument 50, which helps in manufacturing and development. A more accurate calibration of the instrument 50 may also be implemented. Calibration may be performed during manufacture, prior to surgery, in the port during insertion, or during surgery. In addition, the optical position determination may be used to determine while the instrument 50 is inside a patient during a procedure. The image sensors 302a-c may be positioned inside the patient and held stationary by other robotic arms 40a-d and/or access ports 55a-d allowing for continuous monitoring of the position of the end effector 50 and/or the jaws 120 and 122 in the manner described above using the imaging assembly 300.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
an instrument including a shaft defining a longitudinal axis and an end effector pivotable relative to the shaft at a yaw angle, the end effector including a pair of jaws pivotable at a pitch angle and openable to a jaw angle;
a first imaging device configured to obtain a first image of the end effector along a first axis;
a second imaging device configured to obtain a second image of the end effector along a second axis; and
a third imaging device configured to obtain a third image of the end effector along a third axis, wherein each of the first, second, and third axes are transverse relative to each other.

2. A surgical robotic system according to claim 1, wherein the first imaging device is configured to obtain the first image of the end effector for determining the yaw angle,
the second imaging device is configured to obtain the second image of the end effector for determining at least one of the pitch angle or the jaw angle, and
the third imaging device is configured to obtain the third image of the end effector for determining at least one of the pitch angle or the jaw angle.

3. The surgical robotic system according to claim 1, wherein the end effector is pivotable relative to the shaft about a first pin defining a first pivot axis.

4. The surgical robotic system according to claim 3, wherein the first imaging device is disposed coaxially with the first pivot axis that is perpendicular to the longitudinal axis.

5. The surgical robotic system according to claim 3, wherein the third imaging device is disposed perpendicular to the longitudinal axis and the first pivot axis.

6. The surgical robotic system according to claim 1, wherein the end effector includes a pair of jaws pivotable about a second pin defining a second pivot axis.

7. The surgical robotic system according to claim 1, wherein the second imaging device is disposed coaxially with the longitudinal axis.

8. The surgical robotic system according to claim 1, further comprising:
an imaging assembly configured to couple to the instrument and to secure the first imaging device, the second imaging device, and the third imaging device relative to the end effector.

9. The surgical robotic system according to claim 8, wherein the imaging assembly further including a first mount defining a first plane that is aligned with the longitudinal axis and a second mount coupled to the first mount, the second mount defining a second plane that is transverse to the first plane.

10. The surgical robotic system according to claim 1, further comprising:
an image processing device configured to process the first image, the second image, and the third image and to calculate the yaw angle, the pitch angle, and the jaw angle.

11. The surgical robotic system according to claim 10, wherein the image processing device is further configured to determine position of the end effector or each jaw of the pair of jaws relative to the shaft.

12. The surgical robotic system according to claim 1, wherein each of the first imaging device, the second imaging device, and the third imaging device is an optical comparator.

13. The surgical robotic system according to claim 12, wherein the optical comparator includes a laser backlight.
